(19) United States
(12) Patent Application Publication
(10) Pub. No.: US 2021/0268101 A1
(43) Pub. Date: Sep. 2, 2021

(54) NOVEL PEPTIDES FOR VACCINATION AND TREATMENT OF 2019-nCoV INFECTIONS

(71) Applicant: Cel-Sci Corporation, Vienna, VA (US)

(72) Inventor: Daniel H. Zimmerman, Bethesda, MD (US)

(73) Assignee: Cel-Sci Corporation, Vienna, VA (US)

(21) Appl. No.: 17/148,266

(22) Filed: Jan. 13, 2021

Related U.S. Application Data
(60) Provisional application No. 63/002,016, filed on Mar. 30, 2020.
(60) Provisional application No. 62/984,245, filed on Mar. 2, 2020.

(51) Int. Cl. A61K 39/215

(52) U.S. Cl. CPC A61K 39/215; C12N 2770/20034; C12N 2770/20022

(57) ABSTRACT

The invention relates to novel peptide immunoconjugates for treatment, prevention, and diagnosis of coronaviral infections in animals and humans. The immunoconjugates can be used alone or in conjunction with vaccine adjuvants, immune system cells, or other immunomodulators.

### SEQUENCE LISTING

This application contains a “Sequence Listing” submitted as an electronic .txt file named “CS-142_Sequence_ST25.txt,” 8 KB in size. The Sequence Listing is incorporated herein by reference.

### FIELD OF THE INVENTION

The invention relates to novel peptide immunoconjugates for treatment, prevention, and diagnosis of coronaviral infections in animals and humans. The immunoconjugates can be used alone or in conjunction with vaccine adjuvants, immune system cells, or other immunomodulators.

### BACKGROUND

For confirmed 2019-nCoV infections, reported illnesses have ranged from infected people with little to no symptoms to people being severely ill and dying. Symptoms can include: Fever, Cough and Shortness of breath. The CDC believes at this time that symptoms of 2019-nCoV may appear in as few as 2 days or as long as 14 after exposure. This is based on what has been seen previously as the incubation period of MERS viruses. (CDC, Jan. 26, 2020) from https://www.cdc.gov/coronavirus/2019-ncov/about/symptoms.html.

2019-nCoV, SARS-CoV and MERS-CoV belong to the Coronaviridae family and are positive-sense, single-stranded RNA (group IV: (+)ssRNA). In contrast Influenza A viruses belong to the Orthomyxoviridae family and are negative-sense, single-stranded RNA viruses (group V:(−)ssRNA).

SARS-CoV encodes at least 4 major structural proteins: spike (S), nuclecapsid (N), membrane (M) and envelope (E) proteins (Holmes & Lai, 1996; Ksiazek et al., 2003; Peiris et al., 2003; van Boheemen et al., 2012). The nucleocapsid (N) and spike (S) proteins of SARS-CoV appear to be the dominant antigens recognized by serum Abs. CD4+ T cell responses against protein N have been observed in SARS patients and an HLA-A2-restrictied cytotoxic T lymphocyte epitope in the S protein has been identified (Wang et al., 2004; Xu & Gao, 2004). Several SARS-CoV N protein epitopes have been identified in mice studies (Gupta et al., 2006; Liu et al., 2006; K. Yang et al., 2009) and (Zhao et al., 2007) and in studies with sera from SARS patients (Drosten et al., 2003; He et al., 2004; Ho et al 2012: Peng et al., 2006). Modjarrad, et al 2019 focused on the S protein in their phase I study of a MERS DNA candidate vaccine looking at safety as well as immunogenicity, including antibodies and T cell responses. However, the N protein was not included and with HSV and influenza A virus, N proteins provide excellent vaccine epitope candidates and are often more conserved. Likewise, the S protein being on the virus surface is more influenced by antigenic drift pressures than others, and likewise is its corresponding cell receptor.

Vaccines or therapeutics aimed at preventing or treating 2019-nCoV infections are needed.

### SUMMARY OF THE INVENTION

The first aspect of the invention is directed to peptide heteroconjugates. In any embodiment, the peptide heteroconjugate can include a peptide of SEQ ID NO's: 1-6 or 13-15 conjugated to an immunomodulatory peptide by a direct bond or a divalent linking group.

In any embodiment, the immunomodulatory peptide can be a peptide of SEQ ID NO: 7.

In any embodiment, the immunomodulatory peptide can be a peptide of SEQ ID NO: 8.

In any embodiment, the peptide heteroconjugate can be SEQ ID NO: 9.

In any embodiment, the peptide heteroconjugate can be SEQ ID NO: 10.

In any embodiment, the peptide heteroconjugate can be SEQ ID NO: 11.

In any embodiment, the peptide heteroconjugate can be SEQ ID NO: 12

In any embodiment, the peptide heteroconjugate can be SEQ ID NO: 16.

In any embodiment, the peptide heteroconjugate can be SEQ ID NO: 17.

In any embodiment, the peptide heteroconjugate can be SEQ ID NO: 18.

In any embodiment, the peptide heteroconjugate can be SEQ ID NO: 19.

In any embodiment, the peptide heteroconjugate can be SEQ ID NO: 20.

In any embodiment, the peptide heteroconjugate can be SEQ ID NO: 21.

The second aspect of the invention is drawn to a composition. In any embodiment, the composition can include at least one peptide heteroconjugate selected from: SEQ ID NO 9, SEQ ID NO 10, SEQ ID NO 11, SEQ ID NO 12, SEQ ID NO 16, SEQ ID NO 17, SEQ ID NO 18, SEQ ID NO 19, SEQ ID NO 20, and SEQ ID NO 21; and an adjuvant.

In any embodiment, the composition can include at least two of the peptide heteroconjugates.

In any embodiment, the at least one peptide heteroconjugate can include SEQ ID NO 9.

In any embodiment, the at least one peptide heteroconjugate can include SEQ ID NO 10.

In any embodiment, the at least one peptide heteroconjugate can include SEQ ID NO 11.

In any embodiment, the at least one peptide heteroconjugate can include SEQ ID NO 12.

### BRIEF DESCRIPTION OF THE DRAWINGS

FIG. 1 shows a BLAST alignment between 2019-nCoV N protein (GenBank: QHD43423.2) (“Query”) and SARS-CoV N protein (GenBank: ACZ72117.1) (“Sbjct”).

FIG. 2 shows a BLAST alignment between 2019-nCoV N protein (GenBank: QHD43423.2) (“Query”) and MERS-CoV N protein (GenBank: AFS88943.1) (“Sbjct”).

FIG. 3 shows a BLAST alignment between SARS-CoV N protein (GenBank: ACZ72117.1) (“Query”) and MERS-CoV N protein (GenBank: AFS88943.1) (“Sbjct”).

FIG. 4 shows a BLAST alignment between SARS-CoV S protein (GenBank: AAR86775.1) (“Query”) and MERS-CoV S protein (GenBank: AGN52936.1) (“Sbjct”).

FIG. 5 shows a table of an in silico analysis of several epitopes.

FIGS. 6A-B show the setup of studies to determine the effectiveness of peptide heteroconjugates.

FIGS. 7A-D show results of a prophylaxis study using selected peptide heteroconjugates given 4 and 2 weeks before virus challenge showing changes in individual animal weights.

FIGS. 8A-D show results of a treatment study using selected peptide heteroconjugates given 24 hours after viral challenge showing changes in individual animal weights.

FIG. 9 shows a Kaplan Meier survival plot when selected peptide heteroconjugates are used either for treatment or prophylactically.

### DETAILED DESCRIPTION OF THE INVENTION

Unless defined otherwise, all technical and scientific terms used herein generally have the same meaning as commonly understood by one of ordinary skill in the relevant art.

The articles “a” and “an” are used herein to refer to one or to more than one (i.e., to at least one) of the grammatical object of the article. For example, “an element” means one element or more than one element.

The term “adjuvant” refers to substance that accelerates, prolongs, or enhances antigen-specific immune responses when used in combination with vaccine antigens.

The term “comprising” includes, but is not limited to, whatever follows the word “comprising.” Use of the term indicates the listed elements are required or mandatory but that other elements are optional and may or may not be present.

The term “consisting of” includes and is limited to whatever follows the phrase the phrase “consisting of.” The phrase indicates the limited elements are required or mandatory and that no other elements may be present.

The phrase “consisting essentially of” includes any elements listed after the phrase and is limited to other elements that do not interfere with or contribute to the activity or action specified in the disclosure for the listed elements. Thus, the phrase indicates that the listed elements are required or mandatory but that other elements are optional and may or may not be present, depending upon whether or not they affect the activity or action of the listed elements.

### Immunoconjugates

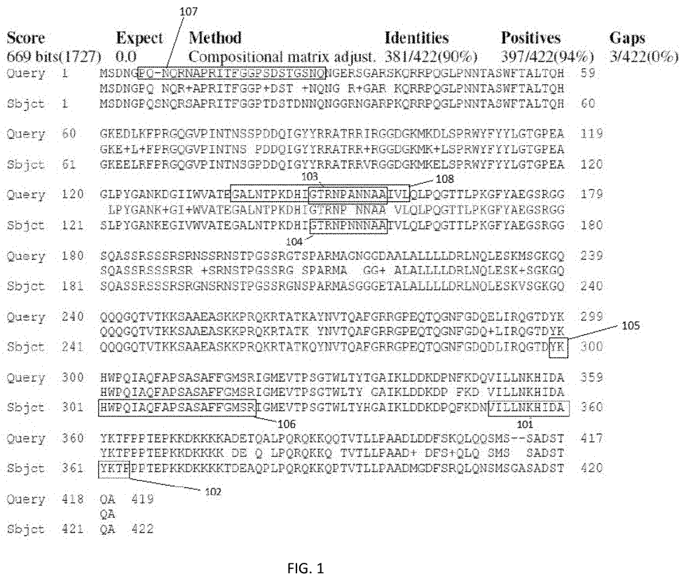

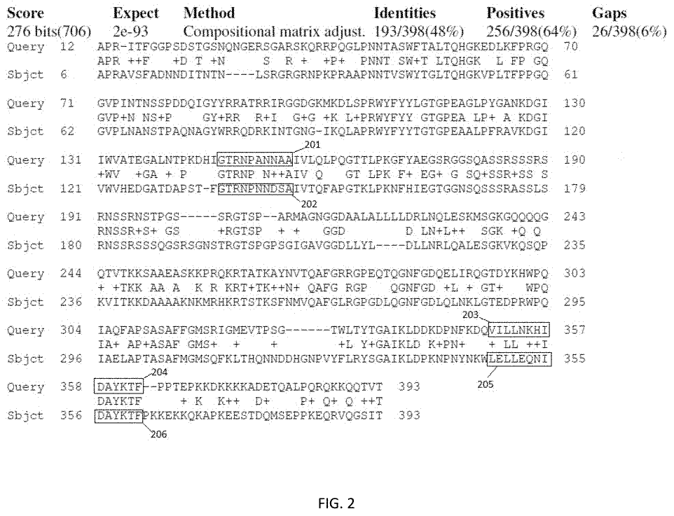

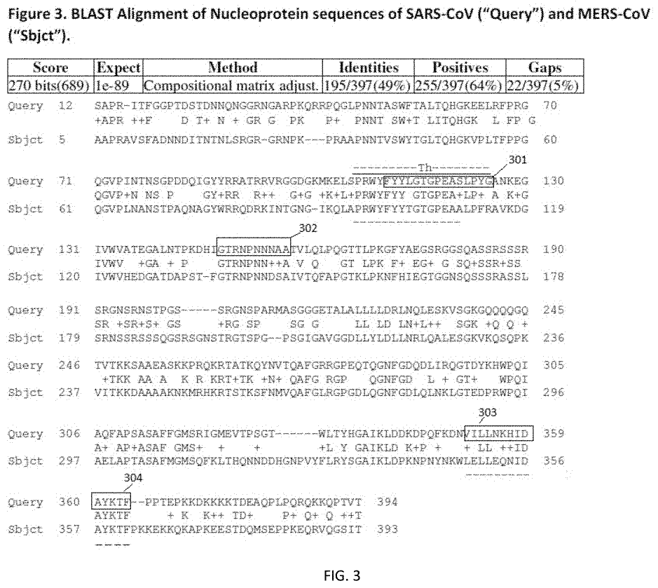

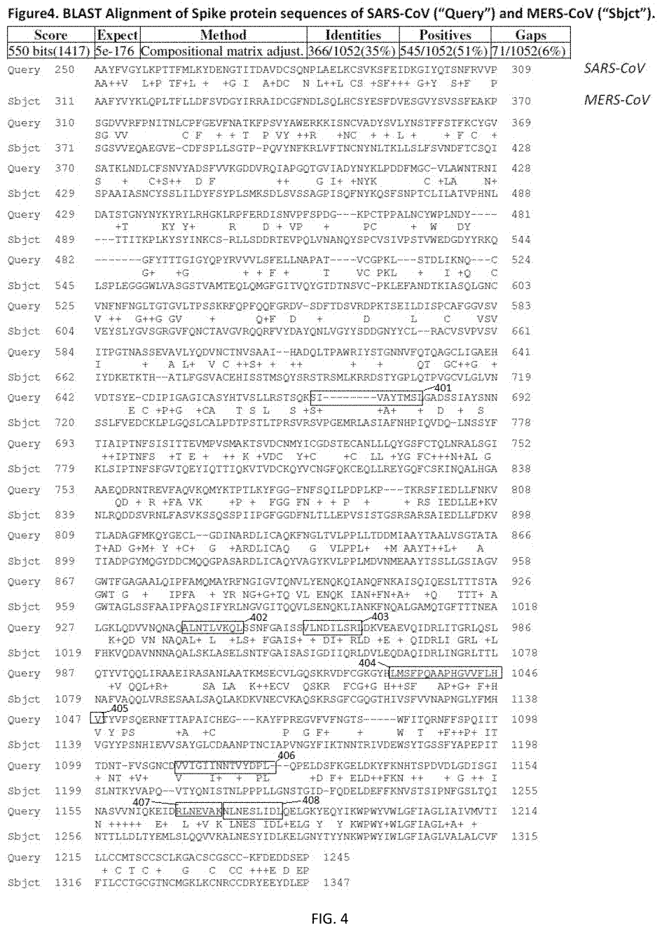

FIGS. 1-2 show BLAST alignments (http://blast.ncbi.nlm.nih.gov/Blast.cgi) between 2019-nCOV N protein (GenBank: QHD43423.2) shown as (“Query”) in FIGS. 1-2 and SARS-CoV N protein (GenBank: ACZ72117.1) and MERS-CoV N protein (GenBank: AFS88943.1), respectively, shown as “sbjct” in FIGS. 1-2. FIG. 3 shows a BLAST alignment between SARS-CoV N protein (GenBank: ACZ72117.1), shown as “Query” and MERS-CoV N protein (GenBank: AFS88943.1), shown as “sbjct”). FIG. 4 shows a BLAST alignment between SARS-CoV S protein (GenBank: AAR86775.1) (“Query”) and MERS-CoV S protein (GenBank: AGN52936.1) (“Sbjct”).

Several areas of overlap appear between the sequences. In particular, N111 and N351 include conserved sequences of several amino acids. Several other amino acids show a full match or a high degree of similarity (i.e. similar aa substitution: eg. Y (tyrosine) for F (phenylalanine) at position 124, with Y having similar structure to F with an additional OH group of the benzene ring. NP111 and NP351 have been identified by Zhao, 2007 as T-helper epitopes (NP111 in C57BL, NP351 in C3H mice) and able to proliferate T-cell lines from lymph node cells. Also, priming with NP111 significantly accelerated the immune response induced by rec NP as indicated by production of NP-specific antibodies. Two epitopes of interest are shown in FIG. 1. Boxes 103, SEQ ID NO. 1, and 104, SEQ ID NO. 2, indicate epitope of interest corresponding to 148-157 aa, as well as box 108 SEQ ID NO 15. The sequence shown in box 101 and continuing in box 102, SEQ ID NO. 3 correspondents to epitope of interest 351-365. Also of interest are the sequence shown in box 107 and the sequence shown in box 105 continuing to box 106.

FIG. 2 shows a BLAST alignment (http://blast.ncbi.nlm.nih.gov/Blast.cgi) between SARS-CoV S protein (GenBank: AAR86775.1) (“Query”) and MERS-CoV S protein (GenBank: AGN52936.1) (“Sbjct”). Unlike the 51 specific HmAbs, the S2 specific HmAbs, including two conserved heptad repeats (HR1:901-1040 aa and HR2:1141-1184 aa) can neutralize pseudotyped viruses expressing different S proteins containing receptor binding domain sequences of various clinical isolates. These data indicate that HmAbs, which bind to conserved regions of the S protein, are more suitable for conferring protection against a wide range of SARS-CoV variants and have implications for generating therapeutic antibodies or subunit vaccines against other enveloped viruses (Elshabrawy et al., 2012). The epitope in box 201, SEQ ID No. 1, is the same as in box 103 of FIG. 1. The epitope of box 202 is SEQ ID No. 4. Box 203 and 204 show sequence ID No. 3, which is the same as boxes 101-102 in FIG. 1. Boxes 205 and 206 show SEQ ID No. 5.

FIG. 3 shows a BLAST alignment between Nucleoprotein sequences of SARS-CoV (“Query”) and MERS-CoV (“Sbjct”). The box labeled 302 is the same sequence as in box 104 of FIG. 1. The box labeled 301 is NP111. The sequences in boxes 303-304 show NP351.

FIG. 4 shows a BLAST alignment of Spike protein sequences of SARS-CoV (“Query”) and MERS-CoV (“Sbjct”). The boxes labeled 401, 402, 403, and 408 indicate SARS-CoV-S epitopes in the literature not, or poorly, conserved in MERS-CoV-S: 678-681, 940-948, 958-966 (Lv, 2009). The boxes labeled 404-405 and 406 also indicate SARS-CoV-S epitopes in the literature not, or poorly, conserved in MERS-CoV-S(Huang et al., 2007). The box labeled 407 is a potential candidate 1167-1175.

Based on the BLAST alignments, several epitopes of interest have been identified. With respect to NP-351, SARS-CoV-N 351-365, mapping of antigenic sites on the SARS-CoV-N protein by ELISA against the sera of 42 patients showed reactivity of immune sera with peptide 354-370 aa being positive in >50%. (He et al., 2004). Peptide 346-362 tested 75% (n=8) positive and peptide 354-370 tested 40% (n=5) positive for production of IFN-γ by PBMCs from SARS—recovered donors in response to single SARS-CoV N peptide, by ELIspot (L.-T. Yang et al., 2006). Studies have also shown that peptide 147-162>40%, peptide 153-170>80% response by ELISA against the sera of 42 SARS patients (He, 2004).

The third identified epitope of interest is SARS-CoV-S-1167-1182 (52 domain): RLNEVAKNLNESLIDL (S1167). The fourth epitope of interest is SARS-CoV-S-791-805 (S2 domain): PLKPTKRSFIEDLLF (S791).

FIG. 5 is a table of an in silico analysis of several epitopes identified by Ho et al 2012 conducted on several different occasions as various alerts arose for SARA and MERS once the original tables are located. In consideration of epitopes to use in LEAPS conjugates besides the table in FIG. 5 of past experience for viruses and LEAPS, the location and epitope size were also considered. Preferably, the immunoconjugates use peptides larger than the basic minimal epitope of around eight or 9 residues so as to encompass overlapping epitopes located in highly conserved regions of essential proteins, to be in a position more likely to minimize effects of influenza virus A antigenic drift.

Based upon the information from the in silico analysis and on epitopes of Covid2019 and related MERS and SARS proteins, immunogenicity studies and potential vaccine candidates, the following 10 LEAPS candidates representing distinct N protein epitopes NP350 “VILLNKHIDAYKTF” (SEQ ID No 3), NP 146 “IGTRNPANNAAIVLP” (SEQ ID No 6), NP6 “PQNQRNAPRITFGGPSDSTGSNQ” (SEQ ID No 13), NP298 “YKHWPQIAQFAPSASAFFGMSR” (SEQ ID No 14), and NP137 “GALNTPKDHIGTRNPANNAAIVL” (SEQ ID No 15) were considered for initial evaluation. Each epitope can be paired as both a DerG (SEQ ID No 7) and a J (SEQ ID No 8) LEAPS conjugate for evaluation purposes for immunogenicity, or in therapeutic purposes. The selection process is similar to previous studies where pairs of J and G [or DerG] epitope conjugates were studied (Rosenthal, et al 1999 with ICP27 epitope for HVS-I efficacy study; Goel, et al 2005 for HSV gD1 epitopes; and Boonnak et al 2013 study with NP protein influenza A virus efficacy studies). For therapeutic purposes, the peptide conjugate pairs can be used alone or with an adjuvant such as ISA51vg. Without being tied to any particular theory of invention, it is believed that J peptide constructs induce a Th1 response, while DerG peptide constructs induce a Th2 response. J-LEAPS vaccines may activate protective T cell responses to CD8 T cell epitopes without need for antibody production, though Th1 associated antibodies may also be elicited. J-LEAPS vaccines may promote maturation of precursors to DCs producing IL12 to direct T cells to initiate antigen-specific Th1 immune responses producing IFNγ. J-LEAPS vaccines can develop protective (anti-viral, anti-cancer) and therapeutic (anti-inflammatory disease, anti-cancer) immune responses.

DerG-LEAPS vaccines may activate Th2 cell responses to CD4 T cell epitopes with antibody production. DerG-LEAPS vaccines may promote Regulatory responses (e.g. anti-inflammatory disease).

J-NP350:
(SEQ ID No 9)
DLLKNGERIEKVEGGGVILLNKHIDAYKTF
J-NP146:
(SEQ IND no 10)
DLLKNGERIEKVEGGGIGTRNPANNAAIVLP
J-NP6:
(SEQ ID No 16)
DLLKNGERIEKVEGGGPQNQRNAPRITFGGPSDSTGSNQ
J-NP298:
(SEQ ID No 17)
DLLKNGERIEKVEGGGYKHWPQIAQFAPSASAFFGMSR
J-NP137:
(SEQ ID No 18)
DLLKNGERIEKVEGGGGALNTPKDHIGTRNPANNAAIVL
DerG-N P350:
(SEQ ID NO 11)
DGQEEKAGVVSTGLIGGGVILLNKHIDAYKTF
DerG-NP 146:
(SEQ ID No 12)
DGQEEKAGVVSTGLIGGGIGTRNPANNAAIVLP
DerG-NP6:
(SEQ ID No 19)
DGQEEKAGVVSTGLIGGGPQNQRNAPRITFGGPSDSTGSNQ
DerG-NP298:
(SEQ ID No 20)
DGQEEKAGVVSTGLIGGGYKHWPQIAQFAPSASAFFGMSR
DerG-NP137:
(SEQ ID No 21)
DGQEEKAGVVSTGLIGGGGALNTPKDHIGTRNPANNAAIVL

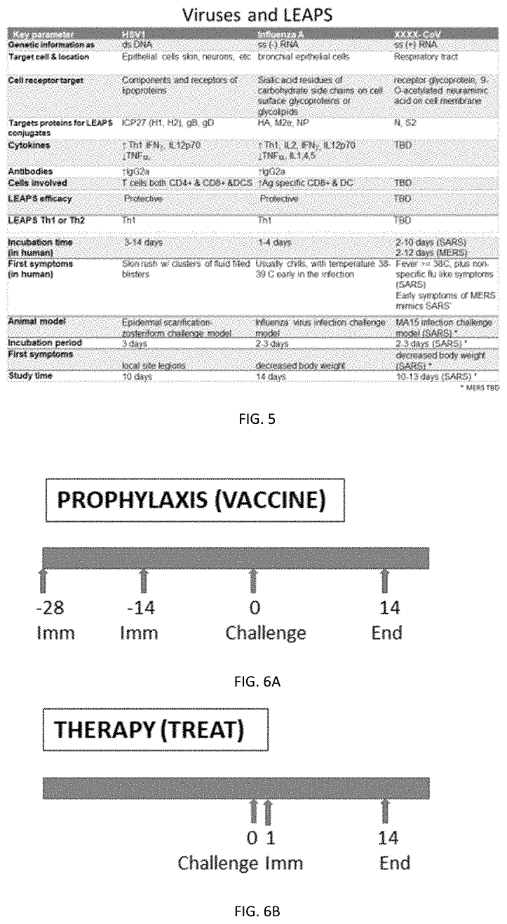

FIGS. 6A-B illustrate studies conducted using four of the conjugates listed above. FIG. 6A illustrates the process of a prophylaxis study to determine the efficacy of a vaccine. Mice were challenged with a lethal dosage of with 2.25×10e+5 pfu/ml SARS-CoV-2 virus. 28 days prior to the challenge, the mice were given a first dose of the vaccine. 14 days prior to the challenge, the mice were given a second dosage of the vaccine. The study was ended 14 days after the challenge, with the mice terminated if weight loss exceeded 25%.

As illustrated in FIG. 6B, for the therapy study, mice were challenged on day 0 with 2.25×10e+5 pfu/ml SARS-CoV-2 virus. One day later, the mice were treated with the peptide constructs. The study ended 14 days after the challenge, with the mice terminated if weight loss exceeded 25%.

Each of the studies illustrated in FIGS. 6A-B used K-18 human ACE-2 expressing transgenic mice on C57Bl/6 background. For each study, the mice were separated into four groups. A first control group received only an adjuvant emulsified with PBS solution in same ratio (1:1); either Seppic ISA51 or PBS. A second control group received a mock vaccine. The mock vaccine is a PBS control solution of NaCl at the same pH and concentration used to dissolve the peptides for the vaccine doses prior to the peptides being mixed and emulsified with an adjuvant. A third group received a pooled dosage of J-NP₃₅₀ (SEQ ID NO 9) and J-NP₁₄₆ (SEQ ID NO 10) in Seppic ISA51. A fourth group received a pooled dosage of DerG-NP₃₅₀ (SEQ ID NO 11) and DerG-NP₁₄₆ (SEQ ID NO 12) in Seppic ISA51.

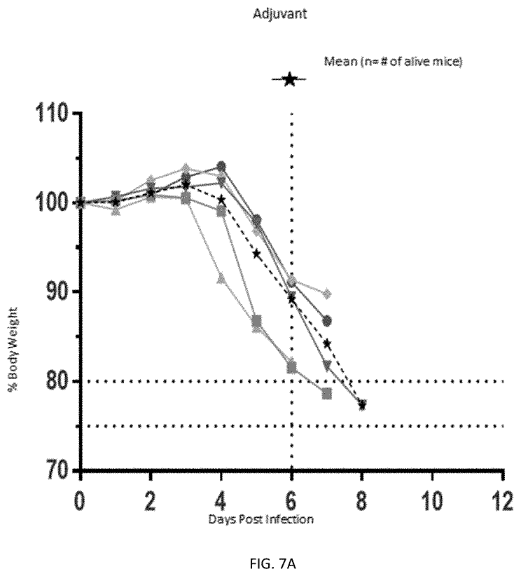

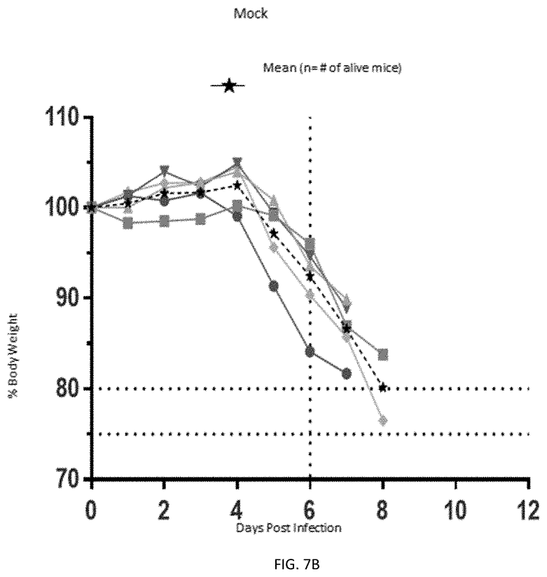

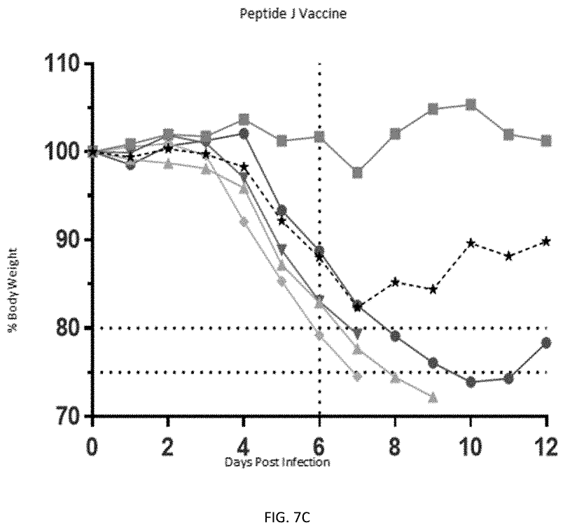

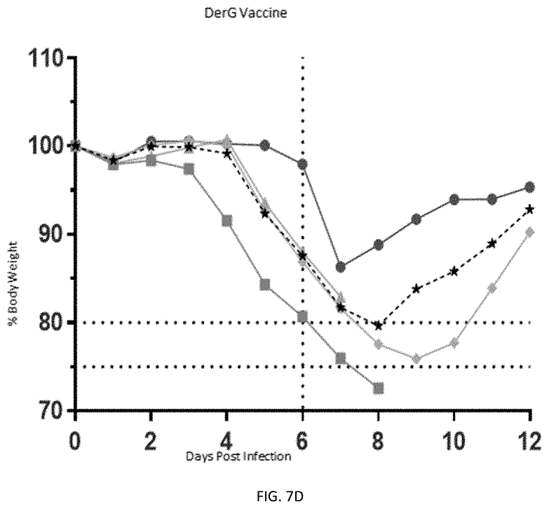

FIGS. 7A-D illustrate the results of the prophylaxis study. FIG. 7A shows the % body weight for each mouse vs. days after infection for mice that received only an adjuvant. FIG. 7B shows the % body weight for each mouse vs. days after infection for mice that received the mock vaccine. FIG. 7C shows the % body weight for each mouse vs. days after infection for mice that received the pooled J-NP₃₅₀ (SEQ ID NO 9) and J-NP₁₄₆ (SEQ ID NO 10) vaccine. FIG. 7D shows the % body weight for each mouse vs. days after infection for mice that received the pooled DerG--NP₃₅₀ (SEQ ID NO 11) and DerG-NP₁₄₆ (SEQ ID NO 12) vaccine. In each of FIGS. 7A-D, the star symbol shows the mean body weight % for all mice still alive.

As illustrated in FIG. 7A, mice that received only an adjuvant experienced significant weight loss. The mean body weight % of the mice reduced to about 77% within 8 days after infection. Similarly, as illustrated in FIG. 7B, the mean body weight % of the mice reduced to about 80% within 8 days after infection.

As illustrated in FIG. 7C, the mice that received the pooled Peptide J vaccines fared better. The mean body weight % of the mice remained above 80% throughout the study, increasing to over 90% within 12 days after infection. As illustrated in FIG. 7D, the mean body weight % of the mice that received the pool DerG vaccines also increased to over 90% within 12 days after infection. All of the control animals that received either mock vaccine or adjuvant only are lost by day 8, and only mice receiving LEAPS conjugates survived past day 8.

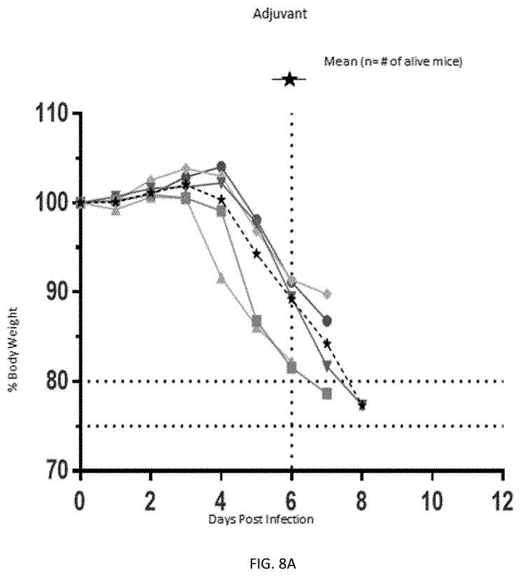

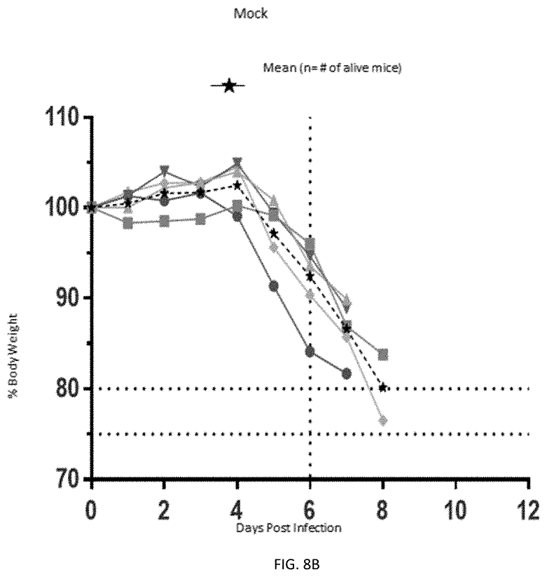

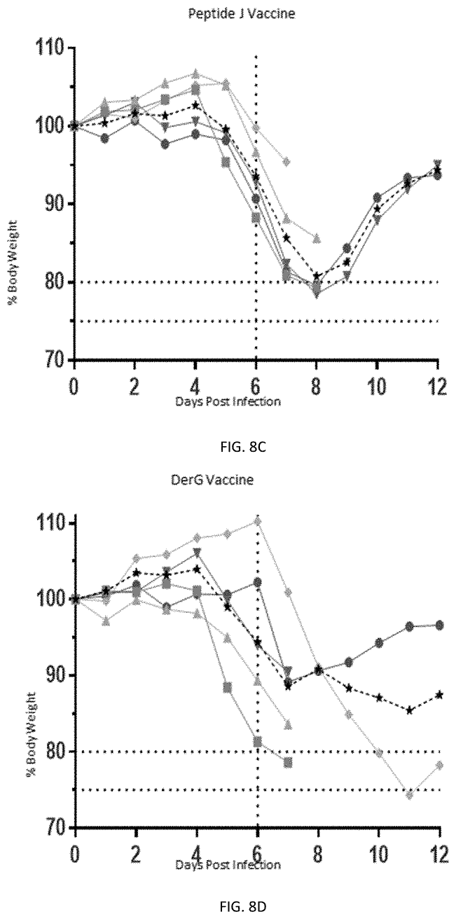

FIGS. 8A-D illustrate the results of the treatment study of LEAPS peptides given 24 hours after infection with SARS-CoV-2 shown in FIG. 6B. FIG. 8A shows the % body weight for each mouse vs. days after infection for mice that received only an adjuvant. FIG. 8B shows the % body weight for each mouse vs. days after infection for mice that received the mock vaccine. FIG. 8C shows the % body weight for each mouse vs. days after infection for mice that received the pooled J-NP₃₅₀ (SEQ ID NO 9) and J-NP₁₄₆ (SEQ ID NO 10) vaccine. FIG. 8D shows the % body weight for each mouse vs. days after infection for mice that received the pooled DerG--NP₃₅₀ (SEQ ID NO 11) and DerG-NP₁₄₆ (SEQ ID NO 12) vaccine. In each of FIGS. 8A-D, the star symbol shows the mean body weight % for all mice still alive.

Similar to the results from the prophylaxis study, the mice that received only an adjuvant in FIG. 8A and the mice that received the mock vaccine in FIG. 8B experienced significant weight loss. The mean body weight for mice that received only the adjuvant decreased to about 77% within 8 days after infection, and the mean body weight % of the mice reduced to about 80% within 8 days after infection for mice that received a mock vaccine.

As illustrated in FIG. 8C, the mean body weight % of the mice that received the pooled J vaccine remained above 80% throughout the study, increasing to nearly 95% of the initial weight within 12 days after infection. Similarly, as illustrated in FIG. 8D, mice that received the pooled DerG vaccines also fared better. The mean body weight % of the mice that received the pool DerG vaccines remained above 85% also increased to nearly 90% within 12 days after infection. All of the control animals that received either mock vaccine or adjuvant only are lost by day 8, and only mice receiving LEAPS conjugates survived past day 8.

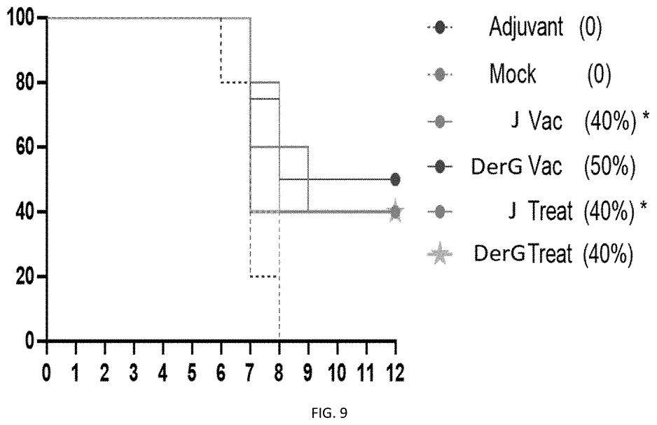

FIG. 9 is a Kaplan Meier survival plot based on the two mouse studies described in FIGS. 6A and 6B. The percentage of mice that survived to 12 days after the challenge in each study is also provided. As illustrated in FIG. 9, all of the mice that received the adjuvant only or the mock vaccine, either prophylactically or after the challenge died within 8 days. However, half of the mice that received the DerG vaccine prophylactically were still alive after 12 days. 40% of the mice that received the peptide J vaccine prophylactically, as well as the mice that received either the DerG vaccine or the peptide J vaccine one day after the challenge were still alive after 12 days.

As illustrated in FIG. 9, almost equivalent protection from SARS-CoV-2 was provided by either the peptide J or DerG LEAPS vaccines administered prophylactically or given as a therapeutic post infection as an immune therapy/treatment. Demonstration of post-infection therapy success is a unique finding, possibly indicating rapid initiation of protection or modulation of immune responses to promote survival in the presence of a pathogenic viral infection.

The demonstration of 40-50% protection following LEAPS administration compared to the 100% mortality of the untreated and only adjuvant treated control animals is an important finding. The results indicate that at least one of the peptides (from the conserved NP region of SARS-CoV2) included in the LEAPS vaccines elicited a protective response. Furthermore, the results indicate that activation of T cells by LEAPS technology is a viable approach to eliciting protection from COVID-19. The current survival results available at twelve days following viral challenge show stabilization or gain of body weight only in the LEAPS immunized animals, while the animals in the control groups lose weight and die much earlier.

Table 1 shows results for an antibody immunogenicity study conducted for each of the four vaccines. The control groups that did not receive any vaccine are not shown. The number of mice that showed antibody response at various concentration cutoffs for each vaccine. Groups of 8 Balb/c mice were immunized with individual conjugates (J-NP₁₄₆, DerG-NP₁₄₆, J-NP₃₅₀, or DerG-NP₃₅₀) as an emulsion in Seppic ISA51 on study days 1 and 14. Serum was obtained on days 10, 19, 28 and 42. Antibodies were assayed at a 1:50 dilution on Neutravidin assay plates coated with Biotin-NP₁₄₆ Biotin-NP₃₅₀ or Biotin-Ova8 (controls).

TABLE 1

| | Proposed | | | | |
|---|---|---|---|---|---|
| | Cutoff OD | | | | |
| | (absorbance | Day 10 | Day 19 | Day 28 | Day 42 |
| Immunogen | at A450) | Responders*/Group Total (n = 8) | | | |
| DerG-NP350 | 0.3 | 8/8 | 8/8 | 8/8 | 8/8 |
| J-NP350 | 0.3 | 1/8 | 7/8 | 8/8 | 8/8 |
| DerG-NP350 | 0.5 | 8/8 | 8/8 | 8/8 | 8/8 |
| J-NP350 | 0.5 | 1/8 | 7/8 | 8/8 | 8/8 |
| DerG-NP350 | 1.0 | 2/8 | 8/8 | 8/8 | 8/8 |
| J-NP350 | 1.0 | 0/8 | 6/8 | 8/8 | 8/8 |
| DerG-NP350 | 1.5 | 0/8 | 8/8 | 8/8 | 8/8 |
| J-NP350 | 1.5 | 0/8 | 1/8 | 2/8 | 6/8 |

| | |
|---|---|
| DerG or J-NP146 | No Antibody detected |

As illustrated in Table 1, all 8 mice in the DerG-NP₃₅₀ group showed antibodies within 10 days at the lower cutoffs of 0.3 and 0.5. Within 19 days, all mice in the DerG-NP₃₅₀ group showed antibodies even at the higher 1.0 and 1.5 cutoffs. One mouse that received the J-NP350 vaccine showed antibodies after 10 days at the lower cutoffs of 0.3 and 0.5. However, 6 of the 8 mice showed antibodies after 19 days at a cutoff of 1.0. Within 42 days after treatment, 6 of the 8 mice showed antibodies even at the higher 1.5 cutoff. No antibodies were detected for mice that received the DerG-NP₁₄₆ or J-NP₁₄₆ vaccines. The absence of antibodies to LEAPS-NP146 peptides may indicate a lack of B cell epitope.

The results illustrated in Table 1, as well as FIGS. 7A-D and 8A-D indicate that the tested vaccines can provide protection prophylactically or as a treatment given after infection has occurred. The vaccines activate T cell responses fast enough to elicit antigen specific protections even if given 1 day after lethal challenge with SARS-CoV-2.

In any embodiment, the described heteroconjugates can be administered to a subject either prophylactically or as a treatment after exposure to SARS-CoV. The heteroconjugates can be administered as a composition. The composition can include at least one of the described heteroconjugates with an adjuvant. Non-limiting examples of adjuvants can include PBS, Seppic ISA51vg, Freund's incomplete adjuvant, Lipid A, MPL, ASO1, AS03, AS04, Novasomes and Liposomes, MF59, QS21, IS01, IS03, IS04, Carbomer 934P, Carbomer 971P, CARBOPOL® 974P, or combinations thereof. Alternatively, other adjuvants known in the art can be used. In certain embodiments, two or more of the described heteroconjugates can be included in the composition, as was used in the prophylactic and treatment studies outlined in FIGS. 6A-B. One of skill in the art will be able to determine the dosage necessary for effective treatment or vaccination.

One skilled in the art will understand that various combinations and/or modifications and variations can be made in the dialysis system depending upon the specific needs for operation. Moreover, features illustrated or described as being part of an aspect of the invention can be included in the aspect of the invention, either alone or in combination.

We claim:

1. A peptide heteroconjugate, comprising:
a peptide of SEQ ID NO's: 1-6 or 13-15 conjugated to an immunomodulatory peptide by a direct bond or a divalent linking group.

2. The peptide heteroconjugate of claim 1, wherein:
the immunomodulatory peptide is a peptide of SEQ ID NO: 7.

3. The peptide heteroconjugate of claim 1, wherein:
the immunomodulatory peptide is a peptide of SEQ ID NO: 8.

4. The peptide heteroconjugate of claim 1, wherein:
the peptide heteroconjugate is SEQ ID NO: 9.

5. The peptide heteroconjugate of claim 1, wherein:
the peptide heteroconjugate is SEQ ID NO: 10.

6. The peptide heteroconjugate of claim 1, wherein:
the peptide heteroconjugate is SEQ ID NO: 11.

7. The peptide heteroconjugate of claim 1, wherein:
the peptide heteroconjugate is SEQ ID NO: 12.

8. The peptide heteroconjugate of claim 1, wherein:
the peptide heteroconjugate is SEQ ID NO: 16.

9. The peptide heteroconjugate of claim 1, wherein:
the peptide heteroconjugate is SEQ ID NO: 17.

10. The peptide heteroconjugate of claim 1, wherein:
the peptide heteroconjugate is SEQ ID NO: 18.

11. The peptide heteroconjugate of claim 1, wherein:
the peptide heteroconjugate is SEQ ID NO: 19

12. The peptide heteroconjugate of claim 1, wherein:
the peptide heteroconjugate is SEQ ID NO: 20.

13. The peptide heteroconjugate of claim 1, wherein:
the peptide heteroconjugate is SEQ ID NO: 21.

14. A composition, comprising:
at least one peptide heteroconjugate selected from the group consisting of: SEQ ID NO 9, SEQ ID NO 10, SEQ ID NO 11, SEQ ID NO 12, SEQ ID NO 16, SEQ ID NO 17, SEQ ID NO 18, SEQ ID NO 19, SEQ ID NO 20, and SEQ ID NO 21; and
an adjuvant.

15. The composition of claim 14, wherein the composition comprises at least two of the peptide heteroconjugates.

16. The composition of claim 14, wherein the at least one peptide heteroconjugate comprises SEQ ID NO 9.

17. The composition of claim 14, wherein the at least one peptide heteroconjugate comprises SEQ ID NO 10.

18. The composition of claim 14, wherein the at least one peptide heteroconjugate comprises SEQ ID NO 11.

19. The composition of claim 14, wherein the at least one peptide heteroconjugate comprises SEQ ID NO 12.


SEQUENCE LISTING (21 sequences)

SEQ ID NO: 1 - protein, 10 aa, Artificial Sequence
  From 2019-nCoV
GTRNPANNAA

SEQ ID NO: 2 - protein, 10 aa, Artificial Sequence
  From SARS-CoV Virus
GTRNPNNNAA

SEQ ID NO: 3 - protein, 14 aa, Artificial Sequence
  From 2019-nCoV Virus
VILLNKHIDAYKTF

SEQ ID NO: 4 - protein, 10 aa, Artificial Sequence
  From MERS-coV Virus
GTRNPNNDSA

SEQ ID NO: 5 - protein, 14 aa, Artificial Sequence
  From MERS-CoV Virus
LELLEQNIDAYKTF

SEQ ID NO: 6 - protein, 15 aa, Artificial Sequence
  From 2019-nCoV virus
IGTRNPANNAAIVLP

SEQ ID NO: 7 - protein, 15 aa, Artificial Sequence
  Synthesized peptide construct
DGQEEKAGVVSTGLI

SEQ ID NO: 8 - protein, 13 aa, Artificial Sequence
  Synthesized peptide construct
DLLKNGERIEKVE

SEQ ID NO: 9 - protein, 30 aa, Artificial Sequence
  Synthesized peptide construct
DLLKNGERIEKVEGGGVILLNKHIDAYKTF

SEQ ID NO: 10 - protein, 31 aa, Artificial Sequence
  Synthesized peptide construct
DLLKNGERIEKVEGGGIGTRNPANNAAIVLP

SEQ ID NO: 11 - protein, 32 aa, Artificial Sequence
  Synthesized peptide construct
DGQEEKAGVVSTGLIGGGVILLNKHIDAYKTF

SEQ ID NO: 12 - protein, 33 aa, Artificial Sequence
  Synthesized peptide construct
DGQEEKAGVVSTGLIGGGIGTRNPANNAAIVLP

SEQ ID NO: 13 - protein, 23 aa, Artificial Sequence
  From 2019-nCoV virus
PQNQRNAPRITFGGPSDSTGSNQ

SEQ ID NO: 14 - protein, 22 aa, Artificial Sequence
  From 2019-nCoV virus
YKHWPQIAQFAPSASAFFGMSR

SEQ ID NO: 15 - protein, 23 aa, Artificial Sequence
  From 2019-nCoV virus
GALNTPKDHIGTRNPANNAAIVL

SEQ ID NO: 16 - protein, 39 aa, Artificial Sequence
  Synthesized Peptide Construct
DLLKNGERIEKVEGGGPQNQRNAPRITFGGPSDSTGSNQ

SEQ ID NO: 17 - protein, 38 aa, Artificial Sequence
  Synthesized peptide construct
DLLKNGERIEKVEGGGYKHWPQIAQFAPSASAFFGMSR

SEQ ID NO: 18 - protein, 39 aa, Artificial Sequence
  Syntesized peptide construct
DLLKNGERIEKVEGGGGALNTPKDHIGTRNPANNAAIVL

SEQ ID NO: 19 - protein, 41 aa, Artificial Sequence
  Synthesized peptide construct
DGQEEKAGVVSTGLIGGGPQNQRNAPRITFGGPSDSTGSNQ

SEQ ID NO: 20 - protein, 40 aa, Artificial Sequence
  Synthesized peptide construct
DGQEEKAGVVSTGLIGGGYKHWPQIAQFAPSASAFFGMSR

SEQ ID NO: 21 - protein, 41 aa, Artificial Sequence
  Synthesized peptide construct
DGQEEKAGVVSTGLIGGGGALNTPKDHIGTRNPANNAAIVL